(19) United States
(12) Patent Application Publication
(10) Pub. No.: US 2006/0079689 A1
(43) Pub. Date: Apr. 13, 2006

(54) Processes for preparing and purifying carbostyril compounds such as aripiprazole and 7-(4-halobutoxy)-3,4-dihydro-2(1H)-quinolinones

(76) Inventors: Vladimir Naddaka, Lod (IL); Michael Brand, RaAnana (IL); Guy Davidi, Even-Yehuda (IL); Eyal Klopfer, Tel Aviv (IL); Irina Gribun, Bat-Yam (IL); Oded Arad, Rehovot (IL); Joseph Kaspi, Givatayim (IL)

Correspondence Address: Martin MOYNIHAN;PRTSI, Inc., P.O. Box 16446, Arlington, VA, 22215, US

(21) Appl. No.: 11/246,279

(22) Filed: Oct. 11, 2005

Related U.S. Application Data
(60) Provisional application No. 60/617,073, filed on Oct. 12, 2004.
(60) Provisional application No. 60/675,444, filed on Apr. 28, 2005.

(51) Int. Cl. C07D 403/02

(52) U.S. Cl. 544/363

(57) ABSTRACT

The present invention provides several improved processes for preparing aripiprazole, wherein the first step comprising reacting 7-HQ with a 1,4-disubstituted-butane in biphasic reaction mixture or in a single phase solvent to obtain a 7-(4-halobutoxy)-3,4-dihydro-(1H)-quinolinone (7-HBQ) and the second step comprising reacting the 7-HBQ and 1-(2,3-dichlorophenyl)piperazine or an acid addition salt thereof in a biphasic reaction medium containing water and a water-immiscible solvent to obtain aripiprazole. Also provided are methods of purifying the 7-HBQs and aripiprazole.

### RELATED APPLICATIONS

The present application claims the benefit of U.S. Provisional Patent Application No. 60/617,073, filed on Oct. 12, 2004, and U.S. Provisional Patent Application No. 60/675,444, filed on Apr. 28, 2005, which are incorporated herein by reference in their entirety.

### FIELD OF THE INVENTION

The present invention relates to carbostyril moiety-containing drugs and to chemical intermediates useful in the preparation thereof, and, more particularly, to processes for preparing and purifying 7-(4-halobutoxy)-3,4-dihydroquinolinones, which are of value as intermediates in the synthesis of aripiprazole, and to processes for preparing and purifying aripiprazole.

### BACKGROUND OF THE INVENTION

Aripiprazole (7-{4-[4-(2,3-dichlorophenyl)-1-piperazinyl]-butoxy}-3,4-dihydro-2(1H)-quinolinone) is represented by formula (I).

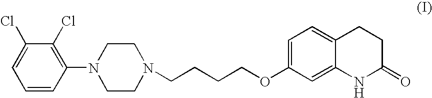

The drug is useful for treating schizophrenia and is available in tablets of different dosages.

Several synthetic methods of aripiprazole preparation are described in U.S. Pat. No. 5,006,528 (hereinafter the '528 patent), including the method illustrated in scheme 1.

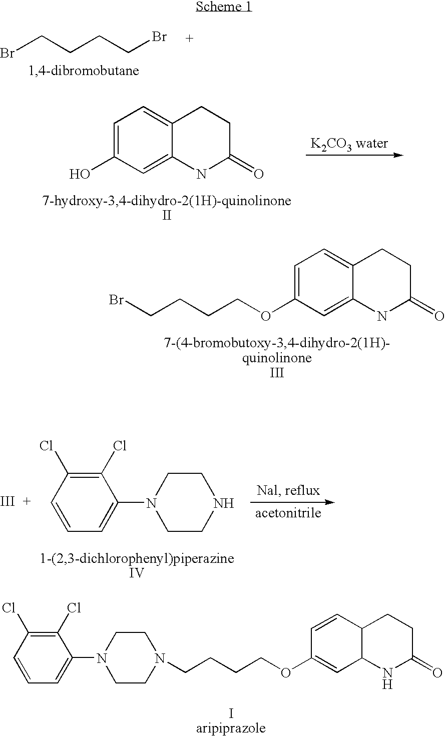

According to this synthetic method aripiprazole is prepared in two steps. The first comprises alkylating the hydroxy group of 7-hydroxy-3,4-dihydro-2(1H)-quinolinone (hereinafter 7-HQ) of formula (II) with 1,4-dibromobutane to obtain 7-(4-bromobutoxy)-3,4-dihydroquinolinone of formula (III) (hereinafter 7-BBQ). A mixture of potassium carbonate, 7-HQ and 3 molar equivalents of 1,4-dibromobutane in water is refluxed for 3 hours. The reaction mixture thus obtained is extracted with dichloromethane, dried with anhydrous magnesium sulfate, and the solvent is removed by evaporation. The residue is purified by means of a silica gel column chromatography (eluent:dichloromethane), eluent evaporation and recrystallization from a mixture of ethanol and n-hexane to obtain 7-BBQ in 75.5% yield.

In the second step 7-BBQ is reacted with 1-(2,3-dichlorophenyl)-piperazine of formula IV to obtain aripiprazole. Thus, a suspension of 7-BBQ and sodium iodide in acetonitrile is refluxed for 30 minutes. Triethylamine and 1-(2,3-dichlorophenyl)piperazine are added to the suspension and the reaction mixture is further refluxed for 3 hours. The solvent is then removed by evaporation, and the residue thus obtained is dissolved in chloroform, washed with water and dried over anhydrous magnesium sulfate. The solvent is removed by evaporation, and the residue is re-crystallized twice from ethanol to give aripiprazole having a melting point of 139.0-139.5° C.

We have repeated the 7-BBQ synthetic procedure described in the '528 patent and found that relatively large amounts of impurities were obtained along with 7-BBQ. Among these impurities, the following were identified and isolated:
1. 1,4-bis[3,4-dihydro-2(1H)-quinolinone-7-oxy]butane (BQB) of formula (V);
2. N-(4-bromobutyl)-7-hydroxy-3,4-dihydro-2(1H)-quinolinone of formula (VI);
3. N-(4-bromobutyl)-7-(4-butoxy)-3,4-dihydro-2(1H)-quinolinone of formula (VII).
1.

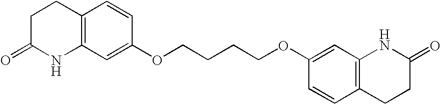

1,4-bis[3,4-dihydro-2(1H)-quinolinone-7-oxy]butane (BQB) (V)
2.

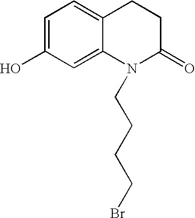

N-(4-bromobutyl)-7-hydroxy-3,4-dihydro-2(1H)-quinolinone (VI)
3.

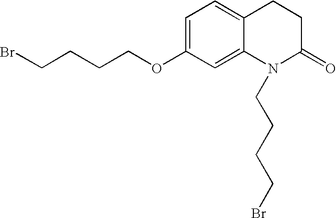

N-(4-bromobutyl)-7-(4-butoxy)-3,4-dihydro-2(1H)-quinolinone (VII)

In a specific run, we have found that 7-BBQ (III) prepared by the above procedure, contained 10% of BQB, which could not be eliminated by re-crystallization, hence the only way to purify 7-BBQ was by column chromatography.

In view of the above mentioned results it is possible that the 75.5% yield of pure 7-BBQ obtained in the '528 patent is overstated.

A second method of preparing 7-BBQ was described by Oshiro Y. et al, J. Med. Chem. 1998, 41, 658-667, wherein 7-BBQ is obtained by reaction of 7-HQ with 3 molar equivalents of 1,4-dibromobutane in N,N-dimethylformamide (DMF) in the presence of potassium carbonate. The reaction is conducted by mixing the reagents for 4 hours at 60° C. followed by diluting with water. Ethyl acetate is added and the layers are separated and the organic phase is washed, dried, and evaporated to dryness in vacuum. Re-crystallization from ethanol gives pure 7-BBQ in 78% yield.

We have repeated this method of preparing 7-BBQ as well, and found that the reaction is very slow in these conditions (only about 40% of 7-BBQ is obtained after 19 hours) and that the reaction mixture contains substantial amounts of BQB.

Another process for preparing Aripiprazole is described in WO 04/063162 and in U.S. Patent Application having the Publication No. 20040192915, which is presented in Scheme 2 below.

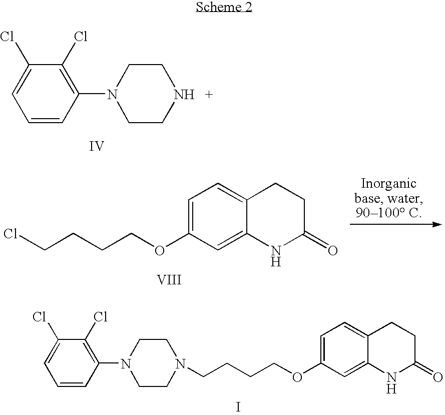

In this process, the N-alkylation of 1-(2,3-dichlorophenyl)piperazine (IV) is carried out with 7-(4-chlorobutoxy)-3,4-dihydro-(1H)-quinolinone (hereinafter 7-CBQ, VIII) in water in the presence of an inorganic base. A mixture of 7-CBQ, 1-(2,3-dichlorophenyl)piperazine mono hydrochloride (1.1 mole equivalents) and potassium carbonate (1.1 mole equivalents) in water (10 vol. with respect to 7-CBQ) is heated with stirring at 90-95° C. for 4 hours. Then, the reaction mixture is cooled to about 40° C., and the obtained crystals are collected by filtration. The crystals are washed with water and dissolved in ethyl acetate (9 vol.), and an azeotropic mixture of water-ethyl acetate (about 3 vol.) is distilled out. The remaining solution is cooled to 0-5° C., and the crystals are collected by filtration and dried to obtain aripiprazole in 92.8% yield having a purity of 99%.

Another process for preparing Aripiprazole is provided in PCT application WO 05/077904 by reacting BBQ and 1-(2,3-dichlorophenyl)piperazine hydrochloride in the presence of a base such as sodium carbonate or potassium carbonate in an organic solvent optionally including a phase transfer catalyst (hereinafter PTC).

However, a need still remains for an improved process of preparing aripiprazole, which will be suitable for large-scale preparation, in terms of simplicity, chemical yield and purity of the product.

### SUMMARY OF THE INVENTION

The present invention provides several improved processes for obtaining aripiprazole.

The first step in the improved process comprises reacting 7-HQ with a 1,4-disubstituted-butane in a water-immiscible organic solvent optionally with addition of substantial amount of water to form an heterogeneous biphasic reaction mixture, in the presence of a water-soluble base and optionally a tetra-alkyl ammonium phase transfer catalyst and a reaction promoter, for a period of time sufficient to completely convert 7-HQ to a 7-(4-halobutoxy)-3,4-dihydro-(1H)-quinolinone (hereinafter 7-HBQ). The 1,4-disubstituted-butane is represented by the general formula of X(CH₂)₄Y, wherein X and Y are independently selected from the group consisting of chlorine, bromine and iodine atoms and a sulfonate.

According to the present invention the first step in the process alternatively comprises preparing the 7-(4-halobutoxy)-3,4-dihydro-2(1H)-quinolinone by alkylating the hydroxy group of 7-HQ with a 1,4-disubstituted-butane in a single liquid phase, in the presence of a base.

Also provided are simple and efficient processes for purifying HBQs, using a purification method selected from the group consisting of slurrying procedure in different solvents, precipitation, and crystallization or a combination of methods thereof, thus the use of column chromatography for purifying the HBQs is avoided.

The second step in the process comprises reacting a 7-HBQ and 1-(2,3-dichlorophenyl)-piperazine or an acid addition salt thereof in a biphasic reaction mixture that comprises water and a water-immiscible solvent in the presence of a base and optionally also a phase transfer catalyst and a reaction promoter.

According to another aspect of the present invention, there are provided methods of purifying aripiprazole, by crystallization or by slurrying procedure in a mixture of a C₁-C₄ alcohol and water.

### DETAILED DESCRIPTION OF THE INVENTION

In a search for alternative processes for preparing 7-{4-[4-(2,3-dichlorophenyl)-1-piperazinyl]-butoxy}-3,4-dihydro-2(1H)-quinolinone (aripiprazole, formula I above), without using column chromatography, the inventors of the present invention have designed and practices improved processes for obtaining aripiprazole as demonstrated in scheme 3 below.

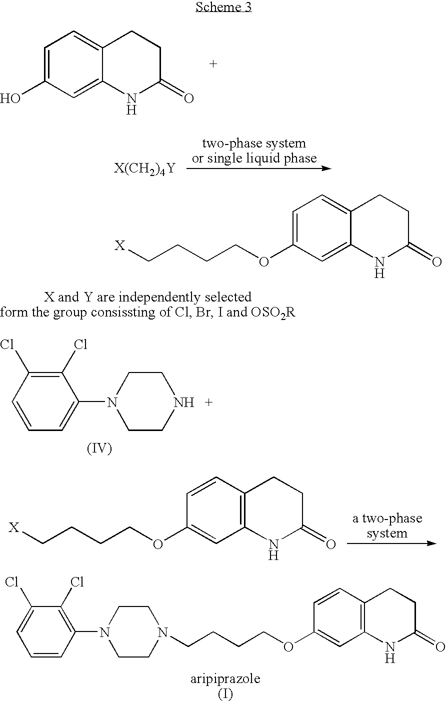

The present invention provides processes for preparing aripiprazole wherein the first step comprises reacting 7-HQ with a 1,4-disubstituted-butane in an organic solvent, optionally with addition of substantial amount of water to form heterogeneous biphasic reaction mixture, containing a water-soluble base and optionally also a tetra-alkyl ammonium phase transfer catalyst and a reaction promoter, for a period of time sufficient to completely convert 7-HQ to a 7-HBQ. The 1,4-disubstituted-butane is represented by the general formula of X(CH₂)₄Y, wherein X and Y are independently selected from the group consisting of chlorine, bromine and iodine atoms and a sulfonate.

According to the present invention the first step of the process alternatively comprises preparing the 7-(4-halobutoxy)-3,4-dihydro-2(1H)-quinolinone by alkylating the hydroxy group of 7-HQ with a 1,4-disubstituted-butane in single liquid phase, in the presence of a base.

Examples of 1,4-disubstituted-butanes in the context of the present invention are 1,4-dichlorobutane, 1-bromo-4-chlorobutane and 1,4-dibromobutane. However, using 1,4-dibromobutane, as taught in the 528′ patent and in PCT application WO 05/077904, is disadvantageous because the compound is a lachrymator, hence its usage might be problematic in industrial scaling-up preparations.

The present inventors have further designed and practiced novel methods of purifying 7-HBQs without using column chromatography selected from the group consisting of slurrying procedure in different solvents, precipitation, and crystallization or a combination of methods thereof.

The purification process can be applied on the 7-HBQ obtained by the processes described herein or by any other process and can be used for obtaining highly pure 7-HBQ and particularly highly pure 7-HBQ that contains a negligible amount of the impurity BQB (see, Formula V above).

Thus, according to the present invention the process for preparing and purifying a 7-HBQ comprising:

a) reacting 7-HQ and a 1,4-disubstituted-butane in an heterogeneous biphasic reaction mixture containing water and a water immiscible organic solvent optionally in the presence of a phase transfer catalyst and a water-soluble base or alternatively in a single solvent phase in the presence of a base to thereby obtain a reaction mixture containing the 7-HBQ;

b) isolating the 7-HBQ from said reaction mixture;

c) optionally purifying the obtained 7-HBQ by precipitation from a suitable organic solvent; and

d) optionally further purifying the obtained 7-HBQ by slurrying the 7-HBQ in an organic solvent and isolating said purified 7-HBQ.

According to the present invention the second step of the said process preferably comprises reacting the 7-HBQ and 1-(2,3-dichlorophenyl)piperazine or an acid addition salt thereof in a biphasic reaction medium that comprises water and a water-immiscible solvent in the presence of a water-soluble base and optionally containing a phase transfer catalyst and a reaction promoter.

Also provided are methods of purifying aripiprazole, by crystallization or by slurrying procedure in a mixture of a C₁-C₄ alcohol and water.

Thus, according to one aspect of the present invention there are provided processes for preparing 7-HBQs, which are carried out by alkylation of the hydroxy group of 7-HQ with a 1,4-disubstituted-butane, optionally in a biphasic reaction mixture in the presence of a base and optionally using a phase transfer catalyst and a reaction promoter, as shown in Scheme 3 above.

While not wishing to be bound by any particular theory, it is assumed that conducting the reaction in a biphasic reaction mixture leads to separation of the base from the base-sensitive 1,4-disubstituted-butane, thus the conversion is increased. It is further assumed that in such conditions the phase transfer catalyst, which is a tetraalkyl ammonium salt, assists in transporting ionic reactants such as a 7-HQ phenolate anion into the non-polar phase and thus its solubility in the water-immiscible organic solvent is increased.

In a preferred embodiment of the present invention there are provided processes for producing 7-(4-halobutoxy)-3,4-dihydro-2(1H)-quinolinone (7-HBQ), wherein a mixture of 7-HQ (II), a 1,4-disubstituted-butane and a base is heated to reflux in a water-immiscible organic solvent, optionally with addition of substantial amount of water, namely in a heterogeneous biphasic reaction mixture, optionally containing a tetra-alkyl ammonium phase transfer catalyst and a reaction promoter. These processes minimize the content of the impurity of formula (V) while producing 7-HBQ in high yield and purity, thus obviating the need to use column chromatography to purify the material. The obtained 7-HBQs may be used for preparing aripiprazole in high quality and yield.

Examples of 1,4-disubstituted-butanes in the context of this preferred embodiment of the present invention are 1,4-dichlorobutane and 1,4-dibromobutane.

The phase transfer catalysts used in the context of this preferred embodiment of the present invention are selected from the group consisting of ammonium salts such as tricaprylylmethylammonium chloride (Aliquate® 336), tetra-n-butylammonium bromide (“TBAB”), tetra-n-butylammonium chloride, tetra-n-butylammonium hydroxide, tetra-n-butylammonium iodide, tetraethylammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethylammonium chloride, and octyltrimethylammonium chloride. Preferred phase transfer catalysts are Aliquate® 336, TBAB, TEBA and combinations thereof. The presently most preferred phase transfer catalyst is Aliquate® 336.

The phase transfer catalyst may be used in a stochiometric or substochiometric amount, preferably from about 0.05 to about 0.25 mol equivalents with respect to 7-HQ.

The base used in these processes may be an inorganic base selected from the group consisting of sodium hydroxide, potassium hydroxide, sodium carbonate, sodium bicarbonate, potassium carbonate, potassium bicarbonate, and combinations thereof. The presently preferred base is potassium carbonate.

Preferably, at least 0.5 molar equivalent of base per 1 mole of 7-HQ is used, and more preferably between 1.1 and 1.3 molar equivalents of base per 1 mole of 7-HQ.

The 1,4-disubstituted-butane may be used in an amount of from about 3 to about 9 molar equivalents with respect to 7-HQ, with about 3 molar equivalents of the 1,4-disubstituted-butane with respect to the 7-HQ being preferable.

The water-immiscible solvent used in these processes is selected from the group consisting of toluene, ethyl benzene, xylenes, and mixtures thereof. The presently most preferred solvent is toluene.

The ratio of water to the water-immiscible solvent can be from about 0.5:1 to about 10:1 (v/v), more preferably from about 1:1 to about 6:1.

In an exemplary embodiment of the process according to this aspect of the present invention, 7-HQ, 1,4-disubstituted-butane, a base and a phase transfer catalyst are mixed in a water-water immiscible solvent mixture and the reaction mixture is heated under reflux until the 7-HQ completely disappears.

The crude 7-HBQ may be isolated from the reaction mixture by separating the organic phase from the aqueous phase followed by removing the organic solvent and the excess of the 1,4-disubstituted-butane by distillation under reduced pressure.

In an alternative exemplary embodiment, the 7-HQ, the base and the phase transfer catalyst are dissolved in water. A 1,4-disubstituted-butane is dissolved in the water-immiscible solvent and the two solutions are mixed and stirred under reflux until the 7-HQ completely disappears.

In another alternative exemplary embodiment, an aqueous suspension of 7-HQ and the phase transfer catalyst is mixed with a solution of the 1,4-disubstituted-butane in the water-immiscible solvent. The biphasic mixture is stirred under reflux, while the base is slowly added to the mixture. The base may be added portion-wise or dropwise as a concentrated aqueous solution of the base.

In yet another alternative embodiment, a biphasic mixture of the water-immiscible organic solvent and the aqueous solution of 7-HQ, the base and the phase transfer catalyst are heated under reflux, while the 1,4-disubstituted-butane is slowly added to the mixture. The 1,4-disubstituted-butane may be added portion-wise or continuously dropwise.

The process according to this aspect of the present invention may further include addition of a reaction promoter, which is soluble in the aqueous phase. Reaction promoters in the context of the present invention are salts of strong acids such as sodium sulfate, which is the salt of sulfuric acid. While not wishing to be bound by any particular theory, it is assumed that sodium sulfate, which is substantially soluble in the aqueous phase, increases the ionic strength of the aqueous solution without modifying the pH of the reaction mixture. Thus it is assumed that sodium sulfate decreases the solubility of 7-HQ in the aqueous phase and as a consequence improves its solubility in the organic phase.

Preferably, the reaction promoter is added in the amount of about 10-20% (w/v) with respect to the aqueous phase.

Using the process described above, the crude 7-HBQ is obtained in high yield and relatively high purity. The only impurity detected is the compound having formula V (BQB) (10-15%).

The 7-HBQ obtained by the processes of the present invention described hereinabove may be used in preparing aripiprazole in good yield and quality, using, for example, the method described in the '528 patent. Thus, a suspension of a 7-HBQ and sodium iodide in acetonitrile is refluxed for 6 hours. 1-(2,3-dichlorophenyl)piperazine and triethylamine are added to the suspension and the reaction mixture is further refluxed for 3 hours. The solvent is than removed by evaporation, and the residue thus obtained is dissolved in chloroform. The solution is washed with water and dried over anhydrous magnesium sulfate. The solvent is removed by evaporation, and the residue thus obtained is re-crystallized twice from ethanol to give aripiprazole having a melting point of 139.0-139.5° C.

In another preferred embodiment of the present invention there are provided processes for producing 7-(4-halobutoxy)-3,4-dihydro-2(1H)-quinolinone (7-HBQ), which are carried out by alkylating the hydroxy group of 7-HQ with a 1,4-disubstituted-butane in a single liquid phase, in the presence of a base.

Examples of 1,4-disubstituted-butanes in the context of this preferred embodiment of the present invention are 1,4-dichlorobutane, 1-bromo-4-chlorobutane and 1,4-dibromobutane.

The 1,4-disubstituted-butane may be used in an amount of from about 3 to about 9 molar equivalents. It may be seen by comparing examples 2 and 5, that when an excess of 3 equivalents of 1,4-dichlorobutane was used the reaction mixture contained about 13.5% of BQB upon reaction completion while when an excess of 9 equivalents of 1,4-dichlorobutane was used, the reaction mixture contained only about 4.5% of BQB upon reaction completion. However, using an excess of raw material is disadvantageous from the economic aspect of the process and therefore using about 3 molar equivalents of the 1,4-disubstituted-butane with respect to the 7-HQ is preferable.

Suitable solvents that may be used as the single liquid phase are selected from the group consisting of acetonitrile, acetone, methyl ethyl ketone, N,N-dimethylformamide (DMF), methanol, ethanol, 1-propanol, 2-propanol, 1-butanol, 2-butanol, isobutanol, and mixtures thereof. The most preferred solvent are 1-propanol, 2-propanol, acetonitrile, and mixtures thereof.

According to present invention, a mixture comprising 7-HQ, a 1,4-disubstituted-butane and a base is heated, preferably under reflux, in the solvent for a time period sufficient to allow complete conversion of 7-HQ to 7-(4-halobutoxy)-3,4-dihydro-2(1H)-quinolinone.

The base used in this process may be an inorganic base selected from the group consisting of sodium hydroxide, potassium hydroxide, sodium carbonate, potassium carbonate, and combinations thereof. The preferable bases in the single liquid phase process are solid potassium carbonate, solid potassium hydroxide and aqueous sodium hydroxide.

Preferably, at least 0.5 molar equivalent of the base per 1 mole of 7-HQ is used, and more preferably between 1.1 and 3 molar equivalents of base per 1 mole of 7-HQ.

The reaction progress may be monitored by using high performance liquid chromatography (HPLC), thus the reaction is stopped after complete disappearance of 7-HQ. After reaction completion the hot reaction mixture is filtered, and the alcohol and an excess of 1,4-dichlorobutane are removed by evaporation to dryness in vacuum. The solid thus obtained is treated with an alcohol to remove the traces of 1,4-disubstituted-butane and the solid is collected by filtration to obtain crude a 7-HBQ.

Using the process described above, the crude 7-HBQ is obtained in high yield and relatively high purity. The only impurity detected was the compound having formula V (BQB) (4.5-15%).

In an exemplary embodiment of the present invention there is provided a process for producing 7-(4-chlorobutoxy)-3,4-dihydro-2(1H)-quinolinone (7-CBQ) of formula (VIII), wherein a mixture of 7-HQ (II), 1-bromo-4-chlorobutane and a base is heated to reflux in a C₁-C₄ alcohol. The process minimizes the content of the impurity of formula (V) while producing 7-CBQ in high yield and purity, thus obviating the need to use column chromatography to purify the material. The obtained 7-CBQ may be used for preparing aripiprazole in high quality and yield.

The reagent 1,4-dibromobutane is a lachrymator and therefore its usage, as taught in the '528 patent and in PCT application WO 05/077904, specially in large-scale industrial application might be problematic. Therefore by using the intermediate 7-CBQ instead of 7-BBQ the usage of 1,4-dibromobutane can be avoided.

The process for producing 7-(4-chlorobutoxy)-3,4-dihydro-2(1H)-quinolinone (7-CBQ) by alkylating the hydroxylic group of 7-HQ (II) with 1-bromo-4-chlorobutane, is detailed in Scheme 4.

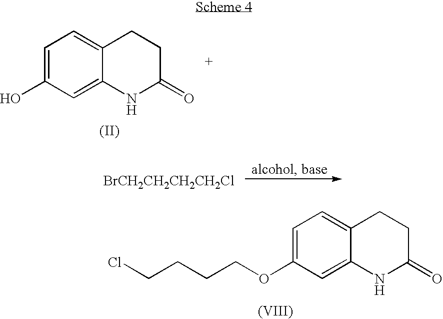

According to the inventive process, a mixture comprising of 7-HQ, 1-bromo-4-chlorobutane and a base is heated preferably under reflux in an organic solvent preferably 1-propanol, 2-propanol, acetonitrile, or a mixture thereof for a time period sufficient to allow complete conversion of 7-HQ to 7-CBQ.

At least 1 molar equivalent of base is used relative to 1 mole of 7-HQ, preferably 1.1-1.3 molar equivalents of base relative to 1 mole of 7-HQ.

More than 1 molar equivalent of 1-bromo-4-chlorobutane relative to 1 mole of 7-HQ is used in the reaction, preferably 3 molar equivalents of 1-bromo-4-chlorobutane relative to 1 mole of 7-HQ.

At least 3 ml of the alcohol relative to 1 g of 7-HQ are used in the reaction, preferably 5 ml of the alcohol relative to 7-HQ.

Using the preparation processes described above, the crude 7-CBQ may obtained from 7-HQ after precipitation from a mixture of 2-propanol and an aqueous basic solution of NaOH in an overall yield of at least 89.2%, preferably of 92.6%, having a purity of at least 98.2%, preferably 98.5%. Thus, 7-HBQ is obtained in high yield and purity without performing column chromatography.

In another aspect, the present invention provides a procedure of purifying a crude 7-(4-halobutoxy)-3,4-dihydro-2(1H)-quinolinone (7-HBQ) without using column chromatography. The procedure, according to this aspect of the present invention, is carried out by slurrying the crude material in a solvent and then isolating the product.

This purification process can be applied on a crude 7-HBQ obtained by any of the processes described hereinabove, as well as on a crude 7-HBQ obtained by any other process.

The solvent used in the slurrying procedure is selected from the group consisting of methyl acetate, ethyl acetate, n-propyl acetate, isopropyl acetate, t-butyl acetate, n-butyl acetate, sec-butyl acetate, isobutyl acetate, toluene, ethyl benzene, xylenes, dichloromethane, chloroform, acetonitrile, acetone, methyl ethyl ketone, methyl isopropyl ketone, methyl propyl ketone, diethyl ketone, t-butyl methyl ketone, methanol, ethanol, 1-propanol, 2-propanol, 1-butanol, 2-butanol, isobutanol, and mixtures thereof. The presently most preferred solvents for the slurrying procedure are methyl acetate, ethyl acetate, acetone, methyl ethyl ketone, toluene, acetonitrile, and methanol.

The slurrying procedure may be carried out at ambient temperature preferably at room temperature during a time period that ranges from about 2 to about 24 hours, preferably during about 8 hours.

Thus, at least 6 ml of methyl acetate relative to 1 gram of the crude 7-HBQ are used in the slurrying procedure, preferably from about 10 ml to about 20 ml of methyl acetate relative to 1 gram of the crude 7-HBQ.

At least 6 ml of ethyl acetate relative to 1 gram of the crude 7-HBQ are used in the slurrying procedure, preferably from about 10 ml to about 20 ml of ethyl acetate relative to 1 gram of the crude 7-HBQ.

At least 4 ml of acetone relative to 1 gram of the crude 7-HBQ are used in the slurrying procedure, preferably from about 8 ml to about 15 ml of acetone relative to 1 gram of the crude 7-HBQ.

At least 5 ml of methyl ethyl ketone relative to 1 gram of the crude 7-HBQ are used in the slurrying procedure, preferably from about 8 ml to about 12 ml of methyl ethyl ketone relative to 1 gram of the crude 7-HBQ.

At least 6 ml of toluene relative to 1 gram of the crude 7-HBQ are used in the slurrying procedure, preferably from about 14 ml to about 20 ml of toluene relative to 1 gram of the crude 7-HBQ.

At least 10 ml of acetonitrile relative to 1 gram of the crude 7-HBQ are used in the slurrying procedure, preferably from about 15 ml to about 25 ml of acetonitrile relative to 1 gram of the crude 7-HBQ.

At least 15 ml of methanol relative to 1 gram of the crude 7-HBQ are used in 30 the slurrying procedure, preferably from about 20 ml to about 25 ml of methanol relative to 1 gram of the crude 7-HBQ.

The progress of the purification process during the slurring procedure may be monitored using HPLC analysis, thus the slurrying procedure is stopped after obtaining a solution that contains less than 2% of BQB. After completing the slurrying procedure the suspension is filtered, and the solvent is removed to dryness in vacuo to obtain the purified 7-HBQ.

Using the preparation and purification processes described above, the 7-HBQ is obtained from 7-HQ in an overall yield of at least 72%, and preferably 75%, and has a purity of at least 98.0%. Thus, 7-HBQ is obtained in high yield and purity without performing column chromatography.

In another aspect, the present invention provides a procedure of purifying a crude 7-HBQ by re-crystallization from an organic solvent comprising:

dissolving 7-HBQ in a suitable organic solvent optionally at elevated temperature to obtain a solution of 7-HBQ in the said suitable solvent; and

isolating the crystals, optionally upon cooling.

The solvent for re-crystallizing may be selected from the group consisting of methanol, ethanol, 1-propanol, 2-propanol, 1-butanol, 2-butanol, acetone, methyl ethyl ketone, diethyl ketone, methyl isobutyl ketone, tert-butyl methyl ether, toluene, methyl acetate, ethyl acetate, isopropyl acetate, butyl acetate, isobutyl acetate, and mixtures thereof. The preferable solvent for re-crystallizing is acetone.

In another aspect, the present invention provides a procedure of purifying a crude 7-HBQ by precipitation from a suitable organic solvent such as 2-propanol. At least 1 ml of 2-propanol relative to Ig of crude 7-CBQ is used in the precipitation procedure, preferably 2 ml of 2-propanol relative to 1 g of crude 7-CBQ are used in the precipitation.

In another preferred embodiment of the present invention the second step of the process for preparing aripiprazole is carried out by reacting 1-(2,3-dichlorophenyl)piperazine or the acid addition salt thereof with a 7-HBQ in a biphasic solvent system comprising a water-immiscible organic solvent and water in the presence of a water-soluble base and a tetra-alkyl ammonium phase transfer catalyst, optionally containing also a reaction promoter as defined hereinabove, for a period of time sufficient to completely convert the 7-HBQ to aripiprazole.

Examples of acid addition salts of 1-(2,3-dichlorophenyl)piperazine that are suitable for use in this process include, without limitation, a mono hydrochloride salt and the dihydrochloride salt.

The 7-HBQ in the context of this preferred embodiment are 7-(4-chlorobutoxy)-3,4-dihydro-2(1H)-quinolinone (7-CBQ) and 7-(4-bromobutoxy)-3,4-dihydro-2(1H)-quinolinone (7-BBQ).

Thus the process for preparing 7-{4-[4-(2,3-dichloro-phenyl)-1-piperazinyl]-butoxy}-3,4-dihydro-2(1H)-quinolinone (aripiprazole) comprising:

a) reacting said 7-HBQ and said 1-(2,3-dichlorophenyl)piperazine or an acid addition salt thereof in a biphasic reaction mixture in the presence of a water-soluble base and optionally also a phase transfer catalyst and a reaction promoter, to thereby obtain a reaction mixture containing the aripiprazole;

b) isolating the aripiprazole from the reaction mixture; and

c) optionally purifying the aripiprazole by slurrying the aripiprazole in a mixture of an organic solvent and water.

In an exemplary embodiment of the present invention, 1-(2,3-dichlorophenyl)piperazine is refluxed with 7-CBQ in a biphasic solvent system containing water and a water-immiscible organic solvent in the presence of a water-soluble base and a tetra-alkyl ammonium phase transfer catalyst for a period of time sufficient to completely convert the 7-CBQ to aripiprazole.

A suitable phase transfer catalyst is selected from a group consisting of ammonium salts such as tetra-n-butylammonium bromide (TBAB), tricaprylylmethylammonium chloride (Aliquate® 336), benzyltriethylammonium bromide (TEBA), tetra-n-butylammonium chloride, tetra-n-butylammonium hydroxide, tetra-n-butylammonium iodide, tetraethylammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethylammonium chloride, and octyltrimethylammonium chloride. Preferred phase transfer catalysts are TBAB, TEBA, Aliquate® 336 and mixtures thereof. The presently most preferred phase transfer catalyst is TBAB.

The phase transfer catalyst may be used in a stochiometric or substochiometric amount, preferably from about 0.05 to about 0.25 molar equivalents with respect to 1-(2,3-dichlorophenyl)piperazine.

The base is preferably an inorganic base selected from the group consisting of sodium hydroxide, potassium hydroxide, sodium carbonate, sodium bicarbonate, potassium carbonate, potassium bicarbonate, and combinations thereof. The presently most preferred base for use in the process according to this aspect of the present invention is potassium carbonate.

Preferably, at least one molar equivalent of the base is used relative to 1 mole of a 1-(2,3-dichlorophenyl)piperazine mono hydrochloride, and more preferably about 1.1 molar equivalent of base relative to 1 mole of 1-(2,3-dichlorophenyl)piperazine mono hydrochloride is used.

Further preferably, at least one molar equivalent of 1-(2,3-dichlorophenyl)piperazine is used relative to 1 mole of the 7-HBQ, and, more preferably, 1.1-1.3 molar equivalents of 1-(2,3-dichlorophenyl)piperazine relative to 1 mole of the 7-BBQ are used.

The water-immiscible solvent is selected from the group consisting of toluene, ethylbenzene, p-xylene, m-xylene, and mixtures thereof. The presently most preferred water-immiscible solvent is toluene.

An excess of water to water-immiscible solvent is preferred, although the ratio may be very wide. Preferred ratios of water to water-immiscible solvent range from about 0.5:1 to about 10:1 (v/v), more preferably from about 1:1 to about 6:1.

According to a preferred embodiment of the process of preparing aripiprazole according to this aspect of the present invention, 7-HBQ, 1-(2,3-dichlorophenyl)piperazine (IV), a base and a phase transfer catalyst are mixed in a water-solvent mixture and the reaction mixture is heated under reflux until the 7-HBQ completely disappears.

In another embodiment, the 1-(2,3-dichlorophenyl)piperazine (IV), the base and the phase transfer catalyst are dissolved in water. The 7-HBQ is dissolved in the water-immiscible solvent and the two solutions are mixed and stirred under reflux until the 7-HBQ completely disappears.

In still another embodiment, an aqueous solution of 1-(2,3-dichlorophenyl)-piperazine and the phase transfer catalyst is mixed with a solution of 7-HBQ in the water-immiscible solvent. The biphasic mixture is stirred under reflux, while the base is slowly added to the mixture. The base may be added portion-wise or dropwise as a concentrated aqueous solution of the base.

In yet another embodiment, a biphasic mixture of the water-immiscible organic solvent and an aqueous solution of 1-(2,3-dichlorophenyl)piperazine, the base and the phase transfer catalyst is heated under reflux, while the 7-HBQ is slowly added to the mixture. The 7-HBQ may be added portion-wise or continuously dropwise.

The process according to this aspect of the present invention can optionally include the addition of a reaction promoter, as described hereinabove, to the aqueous phase, in order to decrease the solubility of 1-(2,3-dichlorophenyl)piperazine in the aqueous phase and to improve the efficiency of its phase transfer to the organic phase. The most preferred reaction promoter is sodium sulfate. Preferably, the reaction promoter is added in about 1-11.1 molar equivalent relative to 1 mole of the 7-HBQ.

Using the process described hereinabove crude aripiprazole can be obtained in 92.6% yield having a purity of 99.2%, as determined by HPLC.

As is mentioned hereinabove, a major limitation associated with the processes for obtaining aripiprazole from 7-HBQ is the presence of the impurity BQB, which is typically obtained during the synthesis of 7-HBQ and is exceptionally difficult to isolate from both the 7-HBQ and the aripiprazole obtained therefrom.

In an exemplary embodiment of the present invention, there is provided a novel method of purifying crude aripiprazole obtained using a 7-BBQ that contains a substantial amount of 1,4-bis[3,4-dihydro-2(1H)-quinolinon-7-oxy]butane (BQB, see, Formula V above) as a reactant. Such crude aripiprazole typically contains by itself a substantial amount of this impurity that is difficult to remove from the final product.

Thus, according to still another aspect of the present invention, there is provided a process of purifying aripiprazole prepared by reacting 7-BBQ that contains at least 10% BQB with 1-(2,3-dichlorophenyl)piperazine. According to this exemplary embodiment of the present invention, crude aripiprazole, containing a substantial amount of BQB, is dissolved in about 18 volumes of a methanol:4% hydrochloric acid (5:4, v/v) mixture, while heating under reflux and the impurity BQB is collected from the hot mixture by filtration. An inorganic base is then added to the hot filtrate to produce a pH of about 10. Methanol is removed from the suspension by distillation, and a colorless precipitate is collected from the hot mixture by filtration and washed with water. The solid is then slurried in a methanol-water (1:1) mixture (5 volumes) for about 6 hours, and the precipitate is collected by filtration from the hot mixture and dried at 60° C. under reduced pressure overnight to give crude aripiprazole (90% yield, containing about 1% of BQB). The crude aripiprazole is slurried twice in methanol (5 volumes) under reflux to give pure aripiprazole (75% overall yield, purity by HPLC: 99.7%; containing less than 0.1% of BQB).

According to another aspect of the present invention, there is provided a method of purifying aripiprazole obtained from a 7-BBQ that contains less than 10% BQB, for example 4% BQB. According to an exemplary embodiment of this aspect, the solid obtained after reaction completion is slurried in a methanol-water (1:1) mixture (5 volumes) for about 6 hours, and the precipitate is collected by filtration from the hot mixture and dried at 60° C. under reduced pressure overnight to give crude aripiprazole (90% yield, containing about 1% of BQB). The crude aripiprazole is slurried twice in methanol (5 volumes) under reflux to give pure aripiprazole (74.5% overall yield, purity by HPLC: 99.7%; containing 0.1% of BQB).

According to another aspect of the present invention, there is provided a process of preparing 1,4-bis[3,4-dihydro-2(1H)-quinolinone-7-oxy]butane (BQB) having a purity of at least 98%, preferably of 99.5%, the process comprising:

a) reacting 7-HQ and a 1,4-dibromobutane in an organic solvent in the presence of a base;

b) isolating the 1,4-bis[3,4-dihydro-2(1H)-quinolinone-7-oxy]butane from the reaction mixture by precipitation; and

c) purifying the product.

The reaction solvent is selected from the group consisting of acetonitrile, acetone, methyl ethyl ketone, N,N-dimethyl-formamide (DMF), methanol, ethanol, 1-propanol, 2-propanol, 1-butanol, 2-butanol, isobutanol, and mixtures thereof. The presently preferred solvent is 1-propanol.

The inorganic base used in the process is selected from the group consisting of sodium hydroxide, potassium hydroxide, sodium carbonate, potassium carbonate, and combinations thereof, preferably potassium hydroxide. The preferable molar ratio used in the process for preparing BQB between 7-HQ, potassium hydroxide and 1,4-dibromobutane is about 2:2:1.

### EXAMPLES

### Example 1

### Preparation of 1,4-bis[3,4-dihydro-2(1H)-quinolinon-7-oxy]butane (BQB)

A mixture of 7-hydroxy-3,4-dihydro-2(1H)-quinolinone (8 g, 0.048 mole, 2 eq.), 1,4-dibromobutane (2.9 ml, 5.24 g, 0.024 mole, 1 eq.) and 85% potassium hydroxide powder (3.22 g, 0.048 mole, 2 eq.) in 1-propanol (40 ml) was heated under reflux for 2 hours. Then, water (20 ml) was added into the hot reaction mixture, the formed precipitate was filtered, washed with hot water (20 ml) and hot 1-propanol (20 ml) and dried at 70° C. overnight to give a crude product (8.5 g). The crude product (8.5 g) was heated under reflux in 1-propanol (130 ml) and sodium hydroxide (45% aqueous solution) was added to the suspension to produce a pH of about 10-11. The reflux was continued during 4 hours and a colorless precipitate was then collected by filtration, washed with water (40 ml) and 1-propanol (40 ml) and dried at 70° C. overnight to give 1,4-bis[3,4-dihydro-2(1H)-quinolinon-7-oxy]butane (7.9 g, purity by HPLC: 99.5%).

Melting point=217.5-218.5° C.

¹H NMR (DMSO-d₆): δ=1.83 (4H, s, —CH₂CH₂—), 2.42 (4H, t, —CH₂CO—), 2.78 (4H, t, —CH₂—C—CO—), 3.95 (4H, s, —O—CH₂—) 6.48 (4H, m, aromatic H), 7.04 (2H, d, aromatic H), 10.0 (2H, s, NHCO).

¹³C NMR (DMSO-d₆): δ=24.0, 25.4, 30.8, 67.1, 101.7, 107.5, 115.5, 128.4, 139.2, 157.8, 170.3.

MS (CI): m/z=381.1 [MH⁺].

Analysis: Calculated for C₂₂H₂₄N₂O₄: % C, 69.43; % H, 6.37; % N, 7.36; Found: % C, 69.35; % H, 6.42; % N 7.41.

### Example 2

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in 1-propanol in the presence of potassium carbonate

A mixture of 7-hydroxy-3,4-dihydro-2(1H)-quinolinone (40 g, 0.245 mole), 1,4-dichlorobutane (97% purity, 82.8 ml, 96.0 g, 0.735 mole. 3 eq.) and potassium carbonate (37.24 g, 0.27 mole, 1.1 eq.) in 1-propanol (400 ml) was heated under reflux for 10 hours (the reaction mixture contained 13.5% of BQB after reaction completion). The hot reaction mixture was then filtered and the solid was washed with hot 1-propanol (3×60 ml). The solvent and the excess of 1,4-dichlorobutane were removed by evaporation in vacuo. 2-Propanol (180 ml) was added to the thus obtained solid and mixing was maintained at 5-10° C. for 3 hours. The solid was then collected by filtration, washed with cold 2-propanol (50 ml) and dried at 50° C. overnight to give crude 7-(4-chlorobutoxy)-3,4-dihydro-2(1H)-quinolinone (56.5 g, 91.0% yield, containing 13% of BQB).

### Purification of crude 7-(4-chlorobutoxy)-3,4-dihydro-2(1H)-quinolinone (7-CBQ)

Slurry in Methyl Acetate:

The crude 7-CBQ (5 g) was slurried in methyl acetate (70 ml) at room temperature for 8 hours. A precipitate (BQB) was collected by filtration and washed with methyl acetate (3×5 ml). Methyl acetate was removed from the filtrate to dryness under reduced pressure and the colorless solid thus obtained was dried at 60° C. overnight to give 7-CBQ (4.2 g, 76.4% total yield, containing 1.6% of BQB).

Melting point=104-105° C.

Slurry in Ethyl Acetate.

The crude 7-CBQ (10 g) was slurried in ethyl acetate (140 ml) at room temperature for 8 hours. A precipitate (BQB) was collected by filtration and washed with ethyl acetate (3×10 ml). Ethyl acetate was removed from the filtrate to dryness under reduced pressure and the colorless solid thus obtained was dried at 60° C. overnight to give 7-CBQ (8.3 g, 75.5% total yield, containing 1.4% of BQB).

Melting point=104-105° C.

Slurry in Acetone:

The crude 7-CBQ (5 g) was slurried in acetone (50 ml) at room temperature for 8 hours. A precipitate (BQB) was collected by filtration and washed with acetone (3×5 ml). Acetone was removed from the filtrate to dryness under reduced pressure and the colorless solid thus obtained was dried at 60° C. overnight to give 7-CBQ (4.1 g, 74.6% total yield, containing 1.9% of BQB). Melting point=104-105° C.

Slurry in Methyl Ethyl Ketone:

The crude 7-CBQ (5 g) was slurried in methyl ethyl ketone (50 ml) at room temperature for 8 hours. A precipitate (BQB) was collected by filtration and washed with methyl ethyl ketone (3×5 ml). Methyl ethyl ketone was removed from the filtrate to dryness under reduced pressure and the colorless solid thus obtained was dried at 60° C. overnight to give 7-CBQ (4.1 g, 74.6% total yield, containing 1.9% of BQB). Melting point=104-105° C.

Slurry in Toluene:

The crude 7-CBQ (5 g) was slurried in toluene (80 ml) at room temperature for hours. A precipitate (BQB) was collected by filtration and washed with toluene (3×10 ml). Toluene was removed from the filtrate to dryness under reduced pressure and the colorless solid thus obtained was dried at 60° C. overnight to give 7-CBQ (4.2 g, 76.4% total yield, containing 1.5% of BQB). Melting point=104-105° C.

Slurry in Acetonitrile:

The crude 7-CBQ (10 g) was slurried in acetonitrile (200 ml) at room temperature for 8 hours. A precipitate (BQB) was collected by filtration and washed with acetonitrile (20 ml). Acetonitrile was removed from the filtrate to dryness under reduced pressure and the colorless solid thus obtained was dried at 60° C. overnight to give 7-CBQ (4.25 g, 77.4% total yield, containing 1.5% of BQB).

Melting point=104.0-105.0° C.

Slurry in Methanol:

The crude 7-CBQ (5 g) was slurried in methanol (110 ml) at room temperature for 8 hours. A precipitate (BQB) was collected by filtration and washed with methanol (3×20 ml). Methanol was removed from the filtrate to dryness under reduced pressure and the colorless solid thus obtained was dried at 60° C. overnight to give 7-CBQ (4.1 g, 74.6% total yield, containing 2.1% of BQB). Melting point=104-105° C.

### Example 3

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in a Mixture of n-propanol and isopropanol in the Presence of potassium carbonate

A reaction vessel charged with a mixture of 7-hydroxy-3,4-dihydro-2(1H)-quinolinone (15 g, 0.092 mole), 1,4-dichlorobutane (30.5 ml, 0.279 mole, 3 eq.) and potassium carbonate (14 g, 0.101 mole) in a mixture of 1-propanol (150 ml) and 2-propanol (75 ml) was heated under reflux for 19 hours (the reaction mixture contained about 15% of BQB after reaction completing). The hot reaction mixture was cooled to 65° C. and filtered. The solid was washed with 1-propanol. The solvent and the excess of 1,4-dichlorobutane were removed by distillation. 2-propanol (75 ml) was added to the residue thus formed and stirring was maintained at room temperature for about 6 hours. The mixture was cooled to 5-10° C., stirring was maintained for about one hour and the solid was separated by filtration. Cold 2-propanol (90 ml) was added to the solid and mixing was maintained at 5-10° C. for 15 minutes. The wet solid (30.8 grams) was then collected by filtration and dried at 60° C. in vacuum to give crude 7-(4-chlorobutoxy)-3,4-dihydro-2(1H)-quinolinone (15.3 g, 66% yield). The mother liquor contained 2.5 grams of BQB).

### Example 4

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in acetonitrile in the Presence of potassium carbonate

A reaction vessel charged with a mixture of 7-hydroxy-3,4-dihydro-2(1H)-quinolinone (3 g, 0.0184 mole), 1,4-dichlorobutane (12.1 ml, 0.110 mole, about 6 eq.) and potassium carbonate (7.6 g, 0.055 mole) in acetonitrile (30 ml) was heated under reflux for 22 hours (the reaction mixture contained about 8.5% of BQB after reaction completion). The hot reaction mixture was then filtered and the solid was washed with hot acetonitrile. The solvent and the excess of 1,4-dichlorobutane were removed by evaporation in vacuum. Methyl tert-butyl ether (MTBE) (20 ml) was added to the solid and mixing was maintained at 5-10° C. for 3 hours. The solid was then collected by filtration, washed with cold MTBE (3×10 ml) and dried at 50° C. overnight to give of crude 7-(4-chlorobutoxy)-3,4-dihydro-2(1H)-quinolinone (3.45 g, 74% yield, containing 6.1% of BQB, purity by HPLC: 91.5%).

### Example 5

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in 1-propanol in the Presence of potassium carbonate

A mixture of 7-hydroxy-3,4-dihydro-2(1H)-quinolinone (20 g, 0.122 mole), 1,4-dichlorobutane (97% purity, 124 ml, 143.8 g, 1.098 mole, 9 eq.) and potassium carbonate (18.6 g, 0.13 mole, 1.1 eq.) in 1-propanol (200 ml) was heated under reflux for 8 hours (the reaction mixture contained 4.5% of BQB after reaction completion). The hot reaction mixture was then filtered and the solid was washed with hot 1-propanol (3×30 ml). The solvent and the excess of 1,4-dichlorobutane were removed from the filtrate to dryness in vacuum. Methyl tert-butyl ether (MTBE) (90 ml) was added to the thus obtained solid and the mixing was maintained at 5-10° C. for 3 hours. The solid was then collected by filtration, washed with cold MTBE (3×20 ml) and dried at 50° C. overnight to give crude 7-(4-chlorobutoxy)-3,4-dihydro-2(1H)-quinolinone (28.8 g, 92.7% yield, containing 4.7% of BQB).

### Example 6

### Preparation of 7-(4-chlorobutoxy)-3,4-dihydro-2(1H)-quinolinone (7-CBQ) by reaction of 7-hydroxy-3,4-dihydro-2(1H)-quinolinone with 1,4-dichlorobutane in the Presence of Phase Transfer Catalyst

A reactor was charged with 7-hydroxy-3,4-dihydro-2(1H)-quinolinone (50 g, 0.307 mole), 1,4-dichlorobutane (97% purity, 103.5 ml, 120 g, 0.921 mol, 3 eq.), potassium carbonate (46.7 g, 0.338 mole, 1.1 eq.), tricaprylylmethylammonium chloride (Aliquat® 336) (5 g), sodium sulfate (30 g), water (250 ml) and toluene (100 ml) and the mixture was heated under reflux for 14 hours. The organic phase was collected and the solvent and an excess of 1,4-dichlorobutane were removed by evaporation in vacuo. 2-Propanol (230 ml) was added to the residue thus obtained and the mixture was stirred at 5-10° C. for 3 hours. A precipitate was then collected by filtration, washed with cold 2-propanol (2×35 ml) and dried at 50° C. overnight to give crude 7-CBQ (70.8 g, 91% yield, containing 14.3% of BQB). The crude 7-CBQ (70.8 g) was slurried in ethyl acetate (990 ml) at room temperature for 8 hours. A precipitate (BQB) was collected by filtration and washed with ethyl acetate (2×50 ml). The ethyl acetate was removed by evaporation under reduced pressure and the colorless solid thus obtained was dried at 60° C. overnight to give 7-CBQ (58.4 g, 75.0% total yield, containing 1.5% of BQB). Melting point=104-105° C.

### Example 7

### Preparation of 7-CBQ by reaction of 7-HQ with 1-bromo-4-chlorobutane in 2-propanol in Presence of About 85% Solid potassium hydroxide

A mixture of 7-HQ (40 g, 0.245 mole), 1-bromo-4-chlorobutane (85.7 ml, 127.5 g, 0.735 mole, 3 eq.) and 85% solid potassium hydroxide (21 g, 0.318 mole, 1.3 eq.) in 2-propanol (200 ml) was heated under reflux for 2 hours. The hot reaction mixture was filtered and the solvent and excess 1-bromo-4-chlorobutane were removed to dryness in vacuum.

2-Propanol (125 ml) was added to the residue thus obtained and the mixture was heated under reflux to obtain a solution. A solution of 47% aqueous sodium hydroxide solution was added to the hot solution to produce a pH of about 10-11 and the mixture was set aside at 10-15° C. for 6 hours. A colorless precipitate was collected by filtration, washed with the cold mixture of water and 2-propanol (1:3, 50 ml) and water (100 ml) and dried under reduced pressure at 50° C. overnight to obtain 7-(4-chlorobutoxy)-3,4-dihydro-2(1H)-quinolinone (56.8 g) in 91.3% yield, having a purity of 98.5% (by HPLC). Re-crystallization from acetone gave colorless needle crystals: mp 104.0-105.5° C.

### Example 8

### Preparation 7-CBQ by a reaction of 7-HQ with 1-bromo-4-chlorobutane in 2-propanol in Presence of potassium carbonate

A mixture of 7-hydroxy-3,4-dihydro-2(1H)-quinolinone (10 g, 0.061 mole), 1-bromo-4-chlorobutane (21.5 ml, 31.88 g, 0.183 mole, 3 eq.) and potassium carbonate (9.25 g, 0.067 mole, 1.1 eq.) in 2-propanol (50 ml) was heated under reflux for 12 hours. The hot reaction mixture was filtered, and the solvent and excess 1-bromo-4-chlorobutane were removed to dryness in vacuum. 2-Propanol (32 ml) was added to the residue thus obtained and the mixture was heated under reflux to obtain a solution. A solution of 47% aqueous sodium hydroxide was added to the hot solution to produce pH of about 10-11 and the mixture was set aside at 10-15° C. for 6 hours. A colorless precipitate was collected by filtration, washed with the cold mixture of water and 2-propanol (1:3, 15 ml) and water (25 ml) and dried under reduced pressure at 50° C. overnight to obtain 7-(4-chlorobutoxy)-3,4-dihydro-2(1H)-quinolinone (14.4 g) in 92.6% yield, having a purity of 98.4% (by HPLC).

### Example 9

### Preparation 7-CBQ by a Reaction of 7-HQ with 1-bromo-4-chlorobutane in 2-propanol in Presence of About 47% sodium hydroxide solution

A mixture of 7-hydroxy-3,4-dihydro-2(1H)-quinolinone (4 g, 0.024 mole) and 1-bromo-4-chlorobutane (8.6 ml, 12.75 g, 0.073 mole, 3 eq.) in 2-propanol (20 ml) was refluxed followed by addition of mixture of about 47% aqueous sodium hydroxide solution (2.5 g, 0.029 mole, 1.2 eq.) and 2-propanol (4 ml). Reflux was continued for 2 hours. The hot reaction mixture was filtered, and the solvent and an excess of 1-bromo-4-chlorobutane were removed to dryness in vacuum.

2-propanol (12.5 ml) was added to the residue thus obtained and the mixture was refluxed to obtain a solution, followed by addition of a solution of 47% aqueous sodium hydroxide to produce a pH of about 10-11. The mixture was set aside at 10-15° C. for 6 hours. A colorless precipitate was collected by filtration, washed with cold mixture of water and 2-propanol (1:3, 7 ml) and water (10 ml). The solid was dried under reduced pressure at 50° C. overnight to obtain 7-(4-chlorobutoxy)-3,4-dihydro-2(1H)-quinolinone (5.55 g) in 89.2% yield, having a purity of 98.2% (by HPLC).

### Example 10

### Preparation of 7-(4-bromobutoxy)-3,4-dihydro-2(1H)-quinolinone (7-BBQ) by Reaction of 7-hydroxy-3,4-dihydro-2(1H)-quinolinone with 1,4-dibromobutane in 1-propanol in the Presence of potassium carbonate

A mixture of 7-hydroxy-3,4-dihydro-2(1H)-quinolinone (10 g, 0.061 mole), 1,4-dibromobutane (22.2 ml, 39.75 g, 0.184 mole. 3 eq.) and potassium carbonate (9.32 g, 0.067 mole, 1.1 eq.) in 1-propanol (100 ml) was heated under reflux for 6 hours (the reaction mixture contained 9.6% of BQB after reaction completion). The hot reaction mixture was then filtered and the solid was washed with hot 1-propanol (3×20 ml). The solvent and the excess of 1,4-dichlorobutane were removed to dryness by evaporation in vacuum. 2-Propanol (50 ml) was added to the residue thus obtained and the mixing was maintained at 5-10° C. for 3 hours. A precipitate was then collected by filtration and dried at 50° C. under reduced pressure overnight to give crude 7-BBQ (16.5 g, 90.7% yield, containing 11.0% of BQB). The crude 7-BBQ (16.5 g) was slurried in ethyl acetate (230 ml) at room temperature for 8 hours. A precipitate (BQB) was collected by filtration and washed with ethyl acetate (3×15 ml). The ethyl acetate was removed to dryness by evaporation under reduced pressure to give 7-BBQ (13.72 g, 75.5% total yield, containing 1.8% of BQB).

Melting point=110-111° C.

### Example 11

### Preparation of 7-(4-bromobutoxy)-3,4-dihydro-2(1H)-quinolinone (7-BBQ) by reaction of 7-hydroxy-3,4-dihydro-2(1H)-quinolinone with 1,4-dibromobutane in the Presence of Phase Transfer Catalyst

A reactor was charged with 7-hydroxy-3,4-dihydro-2(1H)-quinolinone (50 g, 0.307 mole), 1,4-dibromobutane (98% purity, 110.8 ml, 198.7 g, 0.92 mol, 3 eq.), potassium carbonate (55.1 g, 0.399 mole, 1.3 eq.), tricaprylylmethylammonium chloride (Aliquat® 336) (5 g), sodium sulfate (30 g), water (250 ml) and toluene (75 ml) and the mixture was heated under reflux for 2 hours (the reaction mixture contained 12.1% of BQB after reaction completion). The organic phase was collected and the solvent and an excess of 1,4-dibromobutane were removed from to dryness by evaporation in vacuum. 2-Propanol (270 ml) was added to the residue thus obtained and the mixture was stirred at 5-10° C. for 5 hours. A precipitate was then collected by filtration, washed with cold 2-propanol (2×35 ml) and dried at 50° C. overnight to give crude 7-BBQ (82.3 g, 90.0% yield, containing 14.3% of BQB). The crude 7-CBQ (82.3 g) was slurried in ethyl acetate (1150 ml) at room temperature for 8 hours. A precipitate (BQB) was collected by filtration and washed with ethyl acetate (2×100 ml). The ethyl acetate was removed from the filtrate to dryness by evaporation under reduced pressure and the colorless solid thus obtained was dried at 60° C. overnight to give 7-BBQ (65.9 g, 72.0% total yield, containing 1.9% of BQB). Melting point=110-111° C.

### Example 12

### Preparation of aripiprazole by reaction of 1-(2,3-dichlorophenyl)piperazine monohydrochloride with 7-(4-chlorobutoxy)-3,4-dihydro-(1H)-quinolinone in the Presence of Phase Transfer Catalyst and potassium carbonate in a Bi-Phasic Mixture Containing Toluene and Water

A reaction vessel was charged with 7-(4-chlorobutoxy)-3,4-dihydro-(1H)-quinolinone [15.3 g, 0.064 mole], 1-(2,3-dichlorophenyl)piperazine mono hydrochloride (17.8 g, 0.0665 mole), potassium carbonate (9.2 g, 0.0667 mole), tetrabutylammonium bromide (1.8 g), toluene (230 ml) and water (92 ml). The mixture was heated under reflux for 13 hours. Then, the reaction mixture was cooled to about 65° C. and toluene was added (230 ml) and stirring was maintained for 15 minutes. The phases were separated and the aqueous phase was collected (about 96 ml). Water (77 ml) was added to the organic phase and the mixture was stirred at about 65° C. for 15 minutes. The layers were separated and toluene was distilled out (about 184 ml). Ethanol was added (230 ml) in portions at 65° C. to afford a solution. The solution was cooled to about 25° C. and stirred at that temperature for one hour. Then, the solution was cooled to about 5° C. and stirred at that temperature for one hour. The precipitate was collected by filtration and washed with ethanol to obtain a wet solid, which was dried at 60° C. to afford dry crude aripiprazole (17.6 grams, 65% yield), having a purity of 98%. The crude aripiprazole was crystallized twice from ethanol to obtain the crystallized material having a purity of 99.6%

### Example 13

### Preparation of aripiprazole by Reaction of 1-(2,3-dichlorophenyl)piperazine mono hydrochloride with 7-(4-bromobutoxy)-3,4-dihydro-(1H)-quinolinone in the Presence of Phase Transfer Catalyst

A reactor was charged with 7-(4-bromobutoxy)-3,4-dihydro-(1H)-quinolinone (purity: 98%, 50 g, 0.164 mole), 1-(2,3-dichlorophenyl)piperazine mono hydrochloride (48.3 g, 0.180 mole, 1.11 eq.), potassium carbonate (25 g, 0.180 mole, 1.11 eq.), sodium sulfate (25 g), tetra-butylammonium bromide (5 g), toluene (100 ml) and water (250 ml). The mixture was heated under reflux for 1.5 hours. Then, the aqueous phase was collected and water (200 ml) was added to the reaction mixture. The mixture was stirred at room temperature for 2 hours and a precipitate was collected by filtration and washed with water (2×50 ml) and methanol (3×50 ml). The solid was slurried by heating under reflux in a methanol-water (1:1) mixture (370 ml) for 3 hours and the hot suspension was filtered. The colorless crystals were washed with the hot methanol-water (1:1) mixture (3×50 ml) and dried at 60° C. under reduced pressure overnight to give crude aripiprazole (68 g, 92.6% yield with respect to 7-BBQ, purity by HPLC: 99.2%). The crude aripiprazole (68 g) was slurried in methanol (270 ml) under reflux for 6 hours. The hot suspension was then filtered and the crystals were washed with hot methanol (2×50 ml) and dried at 60° C. under reduced pressure overnight to yield aripiprazole (61 g, 83.1% total yield, purity by HPLC: 99.7%). Melting point is 139.0-139.5° C.

### Example 14

### Preparation of aripiprazole by Reaction of 1-(2,3-dichlorophenyl)piperazine mono hydrochloride with 7-(4-bromobutoxy)-3,4-dihydro-(1H)-quinolinone, Containing 14.0% of 1,4-bis[3,4-dihydro-2(1H)-quinolinone-7-oxy]butane (BQB), in the Presence of Phase Transfer Catalyst

A reactor was charged with 7-(4-bromobutoxy)-3,4-dihydro-(1H)-quinolinone [purity: 85.8% (14.0% of BQB), 23.3 g, 0.067 mole], 1-(2,3-dichlorophenyl)-piperazine mono hydrochloride (19.8 g, 0.074 mole, 1.1 eq.), potassium carbonate (10.3 g, 0.074 mole, 1.1 eq.), sodium sulfate (10 g), tetra-butylammonium bromide (2.0 g), toluene (40 ml) and water (100 ml). The mixture was heated under reflux for 1.5 hours. Then, the reaction mixture was cooled and the two layers were separated. The aqueous phase was collected and water (100 ml) was added to the organic phase. The mixture was stirred at room temperature for 2 hours and a precipitate was collected by filtration and washed with water (3×25 ml).

Purification of the Wet Crude Product:

A mixture of the wet crude product, methanol (300 ml) and 4% hydrochloric acid (240 ml) was heated under reflux for 1 hour. The hot mixture was then filtered off to remove the BQB. Sodium hydroxide (46% aqueous solution) was added to the filtrate to produce a pH of about 10, and the solvent was removed from the suspension by distillation and a solid thus formed was collected by filtration. The wet solid was slurried under reflux in a methanol-water (1:1) mixture (150 ml) for 3 hours, followed by cooling the mixture to room temperature. The colorless precipitate thus obtained was collected by filtration and washed with water (3×50 ml). The wet product was slurried in a methanol-water (1:1) mixture (150 ml) at heating under reflux for 6 hours. The hot suspension was then filtered. The product was washed with a hot methanol-water (1:1) mixture (3×50 ml) and dried at 60° C. overnight to give crude aripiprazole (27 g, 90% yield, purity by HPLC: 98.5%, 1.05% of BQB). The crude aripiprazole (27 g) was slurried twice in methanol (135 ml) under reflux for 6 hours. The hot suspension was then filtered and the crystals were washed with hot methanol (2×250 ml) and dried at 60° C. under reduced pressure overnight to yield pure aripiprazole (22.5 g, 75% total yield, purity by HPLC: 99.7%; 0.08% of BQB). Melting point is 139.0-139.5° C.

### Example 15

### Preparation of aripiprazole by Reaction of 1-(2,3-dichlorophenyl)piperazine mono hydrochloride with 7-(4-bromobutoxy)-3,4-dihydro-(1H)-quinolinone, Containing 3.8% of 1,4-bis[3,4-dihydro-2(1H)-quinolinone-7-oxy]butane (BQB), in the Presence of Phase Transfer Catalyst

A reactor was charged with 7-(4-bromobutoxy)-3,4-dihydro-(1H)-quinolinone [purity: 96% (3.8% of BQB), 20.8 g, 0.067 mole], 1-(2,3-dichlorophenyl)piperazine mono hydrochloride (19.8 g, 0.074 mole, 1.1 eq.), potassium carbonate (10.3 g, 0.074 mole, 1.1 eq.), sodium sulfate (10 g), tetra-butylammonium bromide (2.0 g), toluene (40 ml) and water (100 ml). The mixture was heated under reflux for 1.5 hours. Then, the aqueous phase was collected and water (100 ml) was added to the reaction mixture. The mixture was stirred at room temperature for 2 hours and a precipitate was collected by filtration and washed with water (3×25 ml) and methanol (3×20 ml). The wet solid was slurried by heating under reflux in a methanol-water (1:1) mixture (150 ml) for 5 hours and the hot suspension was filtered. The colorless crystals were washed with a hot methanol-water (1:1) mixture (3×25 ml) and dried at 60° C. under reduced pressure overnight to give crude aripiprazole (27.2 g, 90.6% yield, purity by HPLC: 98.4%, 1.2% of BQB). The crude aripiprazole (27.2 g) was slurried twice in methanol (135 ml) under reflux and dried at 60° C. under reduced pressure overnight to yield aripiprazole (22.3 g, 74.5% total yield, purity by HPLC: 99.7%; 0.10% of BQB).

Melting point is 139.0-139.5° C.

Table 1 below summarizes the processes described above for preparing aripiprazole, its intermediate 7-HBQ and the impurity BQB.

TABLE 1

| Experiment | | |
|---|---|---|
| Number | Product | Description |
| 1 | BQB | Reacting 7-HQ, 1,4-dibromobutane, |
| | | K₂CO₃ in 1-propanol |
| 2 | CBQ | Reacting 7-HQ, 1,4-dichlorobutane, |
| | | K₂CO₃ in 1-propanol |
| 3 | CBQ | Reacting 7-HQ, 1,4-dichlorobutane, |
| | | K₂CO₃ in a mixture of 1-propanol |
| | | and 2-propanol |
| 4 | CBQ | Reacting 7-HQ, 1,4-dichlorobutane, |
| | | K₂CO₃ in acetonitrile |
| 5 | CBQ | Reacting 7-HQ, 1,4-dichlorobutane, |
| | | K₂CO₃ in 1-propanol |
| 6 | CBQ | Reacting 7-HQ, 1,4-dichlorobutane, |
| | | K₂CO₃ in toluene, water, Aliquate ® |
| | | 336, Na₂SO₄ |
| 7 | CBQ | Reacting 7-HQ, 1-bromo-4-chlorobutane, |
| | | solid KOH in 2-propanol |
| 8 | CBQ | Reacting 7-HQ, 1-bromo-4-chlorobutane, |
| | | solid K₂CO₃in 2-propanol |
| 9 | CBQ | Reacting 7-HQ, 1-bromo-4-chlorobutane, |
| | | NaOH solution in 2-propanol |
| 10 | BBQ | Reacting 7-HQ, 1,4-dibromobutane, |
| | | K₂CO₃ in 1-propanol |
| 11 | BBQ | Reacting 7-HQ, 1,4-dibromobutane, |
| | | K₂CO₃ in toluene, water, Aliquate ®, |
| | | Na₂SO₄ |
| 12 | Aripiprazole | Reacting 7-CBQ, 1-(2,3-dichlorophenyl)- |
| | | piperazine mono hydrochloride, K₂CO₃ in |
| | | toluene, water, TBAB, Na₂SO₄ |
| 13 | Aripiprazole | Reacting 7-BBQ, 1-(2,3-dichlorophenyl)- |
| | | piperazine mono hydrochloride, K₂CO₃ in |
| | | toluene, water, TBAB, Na₂SO₄ |
| 14 | Aripiprazole | Reacting 7-BBQ, containing 14% BQB, |
| | | 1-(2,3-dichlorophenyl)-piperazine mono |
| | | hydrochloride, K₂CO₃ in toluene, water, |
| | | TBAB |
| 15 | Aripiprazole | Reacting 7-BBQ, containing 3.8% BQB, |
| | | 1-(2,3-dichlorophenyl)-piperazine mono |
| | | hydrochloride, K₂CO₃ in toluene, water, |
| | | TBAB, Na₂SO₄ |

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An improved process for preparing aripiprazole (7-{4-[4-(2,3-dichlorophenyl)-1-piperazinyl]butoxy}-3,4-dihydro-2(1 h)-quinolinone) or a pharmaceutically acceptable salt thereof wherein the first step comprises reacting 7-HQ with a 1,4-disubstuted-butane in a single-phase solvent and in the presence of a base or in a water-immiscible organic solvent optionally with addition of substantial amount of water to form an heterogeneous biphasic reaction mixture, in the presence of a water-soluble base and optionally a tetra-alkyl ammonium phase transfer catalyst and a reaction promoter, for a period of time sufficient to completely convert 7-HQ to a 7-(4-halobutoxy)-3,4-dihydro-(1H)-quinolinone (7-HBQ); and the second step comprises reacting a 7-HBQ and 1-(2,3-dichlorophenyl)piperazine or an acid addition salt thereof preferably in a biphasic reaction mixture that comprises water and a water-immiscible solvent, in the presence of a water-soluble base and optionally also a phase transfer catalyst and a reaction promoter.

2. The process of claim 1, wherein the 1,4-disubstituted-butane is represented by the general formula of X(CH₂)₄Y, while X and Y are independently selected from the group consisting of chlorine, bromine and iodine atoms and a sulfonate.

3. The process of claim 2, wherein the 1,4-disubstituted-butane is selected from the group consisting of 1,4-dichlorobutane, 1-bromo-4-chlorobutane and 1,4-dibromobutane.

4. A process of claim 1 for preparing a 7-(4-halobutoxy)-3,4-dihydro-2(1H)-quinolinone (7-HBQ), the process comprising:
a) reacting 7-HQ and a 1,4-disubstituted-butane in an heterogeneous biphasic reaction mixture containing water and a water immiscible organic solvent in the presence of a phase transfer catalyst and a water-soluble base or in a single liquid phase, in the presence of a base to thereby obtain a reaction mixture containing the 7-HBQ;
b) isolating the 7-HBQ from said reaction mixture;
c) optionally purifying the obtained 7-HBQ by precipitation from a suitable organic solvent; and
d) optionally further purifying the obtained 7-HBQ by slurrying the 7-HBQ in an organic solvent and isolating said purified 7-HBQ.

5. The process of claim 4, wherein said phase transfer catalyst is selected from the group consisting of tricaprylylmethylammonium chloride (Aliquate® 336), tetra-n-butyl-ammonium bromide (TBAB), tetra-n-butylammonium chloride, tetra-n-butylammonium hydroxide, tetra-n-butylammonium iodide, tetraethylammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethylammonium chloride, octyltrimethylammonium chloride, and any combination thereof.

6. The process of claim 5, wherein said phase transfer catalyst is Aliquate® 336.

7. The process of claim 4, wherein the said water-soluble base is an inorganic base selected from the group consisting of sodium hydroxide, potassium hydroxide, sodium carbonate, sodium bicarbonate, potassium carbonate, potassium bicarbonate, and combinations thereof.

8. The process of claim 7, wherein the water-soluble base used in the biphasic solvent system is potassium carbonate and the bases used in the single solvent phase are solid potassium hydroxide, solid potassium carbonate and aqueous sodium hydroxide.

9. The process of claim 4, wherein said water-immiscible solvent used in the biphasic solvent system is selected from the group consisting of toluene, ethylbenzene, p-xylene, m-xylene, and mixtures thereof.

10. The process of claim 4, wherein the solvent used in the reaction comprising the single phase solvent is selected from the group consisting of acetonitrile, acetone, methyl ethyl ketone, N,N-dimethyl-formamide (DMF), methanol, ethanol, 1-propanol, 2-propanol, 1-butanol, 2-butanol, isobutanol, and mixtures thereof.

11. The process of claim 10, wherein the solvents used in the reaction comprising the single phase solvent are 1-propanol, 2-propanol, acetonitrile, and mixtures thereof.

12. The process of claim 4, wherein the obtained 7-HBQ comprises less than 15% of the impurity 1,4-bis[3,4-dihydro-2(1H)-quinolinone-7-oxy]butane (BQB).

13. The process of claim 4, wherein the organic solvent used in the slurrying procedure is selected from the group consisting of methyl acetate, ethyl acetate, n-propyl acetate, isopropyl acetate, t-butyl acetate, n-butyl acetate, sec-butyl acetate, isobutyl acetate, toluene, ethyl benzene, xylenes, dichloromethane, chloroform, acetonitrile, acetone, methyl ethyl ketone, isopropyl methyl ketone, methyl propyl ketone, diethyl ketone, t-butyl methyl ketone, methanol, ethanol, n-propanol, isopropanol, n-butanol, sec-butanol, isobutanol, and mixtures thereof.

14. The process of claim 13, wherein the organic solvents used in the slurrying procedure are methyl acetate, ethyl acetate, toluene, acetonitrile, acetone, methyl ethyl ketone, and methanol.

15. The process of claim 4, wherein the suitable solvent for purifying the obtained 7-HBQ by precipitation is 2-propanol.

16. A method of purifying 7-CBQ by re-crystallizing from an organic solvent comprising:
a) dissolving 7-CBQ in a suitable organic solvent optionally at elevated temperature to obtain a solution of 7-CBQ in the said suitable solvent; and
b) isolating the crystals, optionally upon cooling.

17. The method of claim 16 of purifying 7-CBQ by re-crystallizing, wherein the suitable organic solvent is selected from the group consisting of methanol, ethanol, 1-propanol, 2-propanol, 1-butanol, 2-butanol, acetone, methyl ethyl ketone, diethyl ketone, methyl isobutyl ketone, tert-butyl methyl ether, toluene, methyl acetate, ethyl acetate, isopropyl acetate, butyl acetate, isobutyl acetate, and mixtures thereof.

18. The method of claim 17, wherein the suitable solvent for re-crystallization is acetone.

19. The process of claim 4, wherein said purified 7-HBQ has a purity of at least 98%.

20. The process of claim 4, wherein said purified 7-HBQ contains less than 2% of the impurity BQB.

21. The process of claim 4, wherein the 7-HBQ is obtained in a yield greater than 75% and preferably in a yield equal to or greater than 92.5%.

22. A process of claim 1 for preparing 7-{4-[4-(2,3-dichloro-phenyl)-1-piperazinyl]-butoxy}-3,4-dihydro-2(1H)-quinolinone (aripiprazole), the process comprising:
a) reacting 7-HBQ and 1-(2,3-dichlorophenyl)piperazine or an acid addition salt thereof in a biphasic reaction mixture in the presence of a water-soluble base and optionally also a phase transfer catalyst and a reaction promoter, to thereby obtain a reaction mixture containing the aripiprazole;
b) isolating the aripiprazole from the reaction mixture; and
c) optionally purifying the aripiprazole by slurrying the aripiprazole in a mixture of an organic solvent and water.

23. The process of claim 22, wherein said phase transfer catalyst is selected from the group consisting of tetra-n-butylammonium bromide (TBAB), tricaprylylmethylammonium chloride (Aliquate® 336), benzyltriethylammonium bromide (TEBA), tetra-n-butylammonium chloride, tetra-n-butylammonium hydroxide, tetra-n-butylammonium iodide, tetraethylammonium chloride, benzyltributylammonium bromide, benzyltriethylammonium bromide, tetramethylammonium chloride, cetyltrimethylammonium bromide, cetylpyridinium bromide, N-benzylquininium chloride, hexadecyltrimethylammonium chloride, and octyltrimethylammonium chloride.

24. The process of claim 23, wherein said phase transfer catalyst is TBAB.

25. The process of claim 22 wherein said water-soluble base is an inorganic base selected from the group consisting of sodium hydroxide, potassium hydroxide, sodium carbonate, sodium bicarbonate, potassium carbonate, potassium bicarbonate, and combinations thereof.

26. The process of claim 25, wherein said water-soluble base is potassium carbonate.

27. The process of claim 22, wherein said water-immiscible solvent is selected from the group consisting of toluene, ethylbenzene, p-xylene, m-xylene, and mixtures thereof, preferably toluene.

28. The process of claim 22, wherein the reaction promoter is sodium sulfate.

29. The process of claim 22, wherein the obtained aripiprazole has a purity of at least 99% and preferably equal to or greater than 99.5%.

30. A process of producing a purified aripiprazole, the process comprising:
a) providing a crude aripiprazole obtained by reacting a 7-HBQ and 1-(2,3-dichlorophenyl)piperazine, said 7-HBQ containing at least 10% of the impurity BQB;
b) partially purifying the said crude aripiprazole with a first solvent;
c) isolating the thus obtained partially purified aripiprazole;
d) slurrying the partially purified aripiprazole in a second solvent and re-slurrying in a third solvent; and
e) isolating the thus obtained purified aripiprazole.

31. The process of claim 30, wherein said first solvent is a mixture of methanol and a 4% aqueous HCl solution (5:4, v/v).

32. The process of claim 30, wherein said partially purifying comprises:
a) dissolving said crude aripiprazole in about 18 volumes of said mixture;
b) heating the resulting mixture under reflux;
c) filtering the mixture to thereby obtain said BQB (as a precipitate) and a hot filtrate; and
d) adding said inorganic base to said hot filtrate.

33. The process of claim 30, wherein said second solvent is a methanol-water (1:1, v/v) mixture (5 volumes).

34. The process of claim 30, wherein said re-slurrying is performed twice and the third solvent is methanol.

35. The process of claim 30, wherein said purified aripiprazole has a purity greater than 99.5% and contains less than 0.1% of said BQB.

36. 1,4-bis[3,4-dihydro-2(1H)-quinolinone-7-oxy]butane (BQB) of the formula

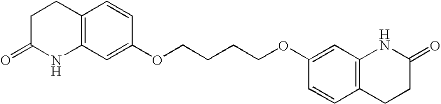

37. A process for preparing 1,4-bis[3,4-dihydro-2(1H)-quinolinone-7-oxy]butane (BQB) having a purity of at least 98%, preferable of 99.5%, the process comprising:
a) reacting 7-HQ and a 1,4-dibromobutane in an organic solvent in the presence of a base;
b) isolating the 1,4-bis[3,4-dihydro-2(1H)-quinolinone-7-oxy]butane from the reaction mixture by precipitation; and
c) purifying the product.

38. The process of claim 37, wherein the reaction solvent is selected from the group consisting of acetonitrile, acetone, methyl ethyl ketone, N,N-dimethylformamide (DMF), methanol, ethanol, 1-propanol, 2-propanol, 1-butanol, 2-butanol, isobutanol, and mixtures thereof.

39. The process of claim 38, wherein the reaction solvent is 1-propanol.

40. The process of claim 37, wherein the base is an inorganic base selected from the group consisting of sodium hydroxide, potassium hydroxide, sodium carbonate, potassium carbonate, and combinations thereof.

41. The process of claim 40, wherein the base is potassium hydroxide, and the molar ratio between potassium hydroxide, 7-HQ and 1,4-dibromobutane is about 2:2:1.
